(19) United States
(12) Patent Application Publication
(10) Pub. No.: US 2018/0296089 A1
(43) Pub. Date: Oct. 18, 2018

(54) APPLICATION FOR SMART PHONE OR RELATED DEVICES FOR USE IN ASSESSMENT OF VESTIBULO-OCULAR REFLEX FUNCTION

(71) Applicants: University of Florida Research Foundation, Incorporated, Gainesville, FL (US); Shands Teaching Hospital and Clinics, Inc., Gainesville, FL (US)

(72) Inventors: Tana B. Carson, Newberry, FL (US); Stephen Z. Sutton, Gainesville, FL (US)

(73) Assignees: University of Florida Research Foundation, Incorporated, Gainesville, FL (US); Shands Teaching Hospital and Clinics, Inc., Gainesville, FL (US)

(21) Appl. No.: 15/569,472

(22) PCT Filed: Apr. 27, 2016
(86) PCT No.: PCT/US2016/029493

§ 371 Date: Oct. 26, 2017

Related U.S. Application Data
(60) Provisional application No. 62/153,389, filed on Apr. 27, 2015.

(51) Int. Cl. A61B 3/113; A61B 3/032; A61B 5/11; G06F 3/01; G06F 3/0346; G06F 3/0482

(52) U.S. Cl. CPC A61B 3/113; A61B 3/032; G06F 3/0482; G06F 3/013; G06F 3/0346; A61B 5/1128

(57) ABSTRACT

A portable and low-cost tool for testing a VOR and oculomotor functions. Such a tool is achieved by programming a smartphone for use in performing such testing. Described herein are computer executable programs for assessing vestibular-ocular reflex including visual acuity and oculomotor function.

### RELATED APPLICATIONS

This application is a National Stage Application of PCT/U.S. 2016/029493, filed Apr. 27, 2016 entitled, “APPLICATION FOR SMART PHONE OR RELATED DEVICES FOR USE IN ASSESSMENT OF VESTIBULO-OCULAR REFLEX FUNCTION”, which claims the benefit under 35 U.S.C. § 119(e) of U.S. Provisional Application 62/153,389, filed Apr. 27, 2015, entitled “APPLICATION FOR SMART PHONE OR RELATED DEVICES FOR USE IN ASSESSMENT OF VESTIBULO-OCULAR REFLEX FUNCTION”, the entire contents of each application which are incorporated herein by reference.

### BACKGROUND

The vestibular system is a sensory organ housed in the inner ear along with the cochlea (the auditory system). The vestibular sensory organ senses angular and linear acceleration of the head and translates that information into neurological impulses. The brainstem, cerebellum and cortical systems use the information about the direction and acceleration of head movement to help maintain upright posture and stable vision. The resulting oculomotor responses to head movement are known as vestibulo-ocular reflexes (VOR). VORs have been well studied in animals as well as both healthy and diseased human populations. Aberrations in a subject's VOR can be indicative of diseases and disorders such as brain tumors/lesions, concussions, neurological disease processes and many other conditions. Several forms of VOR exist; however, rotary VOR is the most widely studied form of VOR, and can be clinically evaluated using dynamic visual acuity (DVA) assessments.

A normal VOR also relies on a functional oculomotor system, a complex sensorimotor network that controls eye movements by incorporating volitional eye movements as well as visual information into appropriate motor outputs to the extra-ocular muscles to direct an individual's gaze. Oculomotor behaviors are also used as indicators of neurological function, assessment of which is relied on by a number of health professions such as neurology, ophthalmology, optometry, primary care, physical and occupational rehabilitation, emergency medicine, sports medicine, and audiology.

Current methods for assessing vestibulo-ocular reflex function require a head piece and/or goggles, a computer and software. Bedside assessments that do not require the above-mentioned equipment do not exist. These methods also require that the subject's head is manually rotated by an examiner and cannot control for the patient resisting head rotation, thus interfering with testing results.

Treatment of vestibulo-ocular reflex and oculomotor conditions is challenging. Home exercises programs are necessary in the rehabilitation process; however, they are often difficult for patients and their families to implement independently. Studies have shown that patients demonstrate better outcomes with feedback regarding the speed of head movements (often requiring encouragement to move faster). Although this feedback can be provided in the clinic, it is difficult to replicate this feedback at home with current exercises programs. A mobile application providing VOR exercises with auditory and/or visual feedback would solve this problem and currently does not exist.

### SUMMARY

Described herein at least one are non-transitory computer storage medium and methods of using said storage medium for the assessment of vestibulo-ocular function and/or visual acuity in a subject. Aspects of the invention provide at least one non-transitory computer storage medium comprising computer executable instructions that, when executed on a portable computing device, controls the computing device to perform a method of assessing dynamic visual acuity (DVA) of a subject. In some embodiments, the methods comprise deriving from sensor data characteristics of head movement of the subject; for each of a plurality of optotypes, the plurality of optotypes having different sizes: when the characteristics meet pre-established criteria, presenting an optotype of the plurality of optotypes on a display of the portable computing device; receiving input indicating the subject's perception of the displayed optotype; based on a comparison of displayed optotypes and received input, recording a smallest optotype of the plurality of optotypes that was accurately reported by the subject.

In some embodiments, the smallest optotype of the plurality of optotypes accurately reported by the subject is compared to a smallest optotype of the plurality of optotypes accurately reported by the subject in a static visual acuity (SVA) assessment. In some embodiments, the difference between the smallest optotype accurately reported by the subject in the dynamic visual acuity (DVA) assessment and the smallest optotype accurately reported by the subject in the static visual acuity (SVA) assessment of 2 or fewer optotype sizes indicates a normal result. In some embodiments, the method further comprises indicating an abnormal result in response to the difference between the smallest optotype accurately reported by the subject in the dynamic visual acuity (DVA) assessment and the smallest optotype accurately reported by the subject in the static visual acuity (SVA) assessment being more than 2 optotype sizes (e.g., 3 or more optotype sizes). In some embodiments, the pre-established criteria of the characteristics of head movement of the subject is rotational movement.

In some embodiments, the rotational movement is at a frequency of about 2 Hertz (e.g., two cycles per second). In some embodiments, the sensor data comprises images acquired by a camera on the portable computing device. In some embodiments, the input is received using a method selected from the list consisting of by video analysis, accelerometer input, manual touch insertion using a touch screen, keyboard, drop down menu, and toggle menu.

Aspects of the invention provide methods for performing a dynamic visual acuity (DVA) assessment comprising positioning a portable computing device in the visual field of a subject at a distance from the subject; detecting movement subject's head; when the subject's head movement meets pre-established criteria, presenting a plurality of optotypes on the display of the portable computing device; receiving information indicating the subject's perception of the displayed optotypes; based on a comparison of displayed optotypes and received input, recording a smallest optotype of the plurality of optotype that was accurately reported by the subject. In some embodiments, the distance is between 2 inches and 20 feet. In some embodiments, the smallest optotype of the plurality of optotypes accurately reported by the subject is compared to a smallest optotype of the plurality of optotypes accurately reported by the subject in a static visual acuity (SVA) assessment. In some embodiments, the difference between the smallest optotype accurately reported by the subject in the dynamic visual acuity (DVA) assessment and the smallest optotype accurately reported by the subject in the static visual acuity (SVA) assessment of 3 or fewer optotype sizes indicates a normal result. In some embodiments, the method further comprises indicating an abnormal result in response to the difference between the smallest optotype accurately reported by the subject in the dynamic visual acuity assessment (DVA) and the smallest optotype accurately reported by the subject in the static visual acuity (SVA) assessment being more than 2 optotype sizes (e.g., 3 or more optotype sizes).

In some embodiments, the portable computing device is maintained in a static position in the subject's field of vision. In some embodiments, the pre-established criteria of the characteristics of head movement of the subject is rotational movement. In some embodiments, the rotational movement is at a frequency of about 2 Hertz (e.g., 2 cycles per second). In some embodiments, the sensor data comprises images acquired by a camera on the portable computing device. In some embodiments, the input is received using a method selected from the list consisting of by video analysis, accelerometer input, manual touch insertion using a touch screen, keyboard, drop down menu, and toggle menu.

Other aspects provide at least one non-transitory computer storage medium comprising computer executable instructions that, when executed on a portable computing device, controls the computing device to perform a method of assessing saccades of a subject. In some embodiments, the method comprises presenting on a display of the portable computing device at least one optotype in different locations at different times thereby defining on the display a first trajectory; processing sensor data to determine movement of the eyes and speed of movement, thereby defining a second trajectory; determining correlation between the first trajectory and the second trajectory. In some embodiments, a duration of eye movement from the optotype at a first location to the optotype at a second location of less than 100 ms indicates a saccade.

Other aspects provide methods for performing a saccade assessment comprising positioning a portable computing device in the visual field of a subject at a distance from the subject; presenting on a display of the portable computing device at least one optotype in different locations at different times thereby defining on the display a first trajectory; detecting movement of the subject's eyes and the speed of movement; receiving information indicating movement of the subject's eyes and speed of the movement, thereby determining a second trajectory; determining the correlation between the first trajectory and the second trajectory. In some embodiments, the portable computing device is maintained in a static position in the subject's field of vision. In some embodiments, the movement of the subject's eyes and the speed of movement is detected by a camera on the portable computing device. In some embodiments, the distance is between 2 inches and 20 feet.

Still other aspects provide at least one non-transitory computer storage medium comprising computer executable instructions that, when executed on a portable computing device, controls the computing device to perform a method of assessing smooth pursuits of a subject. In some embodiments, the method comprises presenting on a display of the portable computing device a moving optotype; processing sensor data to determine movement of the subject's eyes and speed of movement. In some embodiments, the portable computing device is maintained in a static position in the subject's field of vision. In some embodiments, the movement of the subject's eyes and the speed of movement is detected by a camera on the portable computing device. In some embodiments, the distance is between 2 inches and 20 feet.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A and 1B show an example dynamic visual acuity (DVA) assessment.

FIGS. 2A and 2B show an example static visual acuity (SVA) assessment.

FIGS. 3A and 3B show an example saccades assessment.

FIG. 4 shows an example smooth pursuit assessment.

FIG. 5 is a block diagram of a representative computing environment that may execute a visual acuity assessment.

### DETAILED DESCRIPTION

The inventors have recognized and appreciated that a smart phone can be adapted to provide, in a low cost and portable way, VOR and oculomotor testing and treatment. Accordingly, described herein are methods and computer executable applications for adapting a smart phone or related portable computing device for assessment and/or treatment of vestibulo-ocular reflex (VOR) function in the form of dynamic visual acuity (DVA), as well as static visual acuity (SVA) and oculomotor function in a subject. Such an application may improve the accessibility and portability of VOR and oculomotor assessments and allow for early intervention following injury or onset of a neurological disease or disorder and accurate evaluation of treatment progress, for example in the fields of sports medicine, neurology, general medicine and rehabilitative medicine for evaluating oculomotor and vestibular function.

The application may include at least four general functions as follows: (1) static visual acuity (SVA) test, (2) dynamic visual acuity (DVA) test, (3) oculomotor screening assessments for saccades and smooth pursuits and/or (4) treatment exercises. When executed on a portable computing device associated with a user, the application provides a visual stimulus on the display of the portable computing device and receives input information in the form of characteristics of head movement of the subject or the movement, rate, and trajectory of movement of a subject's eyes. The application may also receive additional input from the user, for example results from a static visual acuity (SVA) test or commands to modify the settings for head movement acceleration limits. During dynamic visual acuity (DVA) assessment and treatment, the application will use on the input information such as head acceleration settings to determine if the subject is moving his/her head at a sufficient rate. Thus, the application will determine when to present the optotypes based on this information to ensure that the subject's head is moving sufficiently fast enough to properly test VOR function. The application provides an evaluation of the vesitbulo-ocular reflex or oculomotor function of the subject. In contrast to other available technology, the invention described herein does not require additional equipment such as a headset, goggles, computers, or monitors. However, it is compatible with additional technology or equipment to provide additional input about head, body or eye movements.

### Vestibulo-ocular Reflex (VOR)

The vestibular sensory organ senses angular and linear acceleration of the head and translates that information into neurological impulses. The brainstem, cerebellum and cortical systems then use that information about the direction and acceleration of head movement to help maintain upright posture and stable vision. The resulting oculomotor responses to head movement are known as vestibulo-ocular reflexes (VOR). Horizontal rotary VOR, a clinically relevant and widely studied form of VOR, occurs when the head rotates about the yaw axis (side to side motion). The head rotation stimulates the vestibular system and elicits a compensatory eye movement in the direction opposite of the head movement. This form of VOR enables one to maintain stable vision during movement of the head and body and is required for any daily activities that involve both movement and visual acuity (e.g., reading signs while walking, reaching for a visual target while moving). A functionally relevant measure of rotary VOR function is the test of dynamic visual acuity (DVA).

As used herein, “visual acuity” refers to the clarity of vision and is dependent on several physiological factors such as the ability for the lens of the eye to focus light on the retina, the health of the retina, the neural properties of the retina and optic nerve as well as one's perception or higher order cortical processing of the image. Visual acuity includes both dynamic visual acuity and static visual acuity.

### Dynamic Visual Acuity (DVA)

Dynamic visual acuity (DVA) is a functional measure of the ability for the VOR to maintain stable vision during head movement. Because DVA is critical for vision during motion, defects in DVA can be debilitating. Current methods for testing DVA include two types of tests, both of which require the use of additional equipment (e.g., a headset, goggles, computers, or monitors). The first DVA test involves the use of a plurality of optotypes for determining visual acuity during head movement in the yaw plane (DVA). During performance of a standard DVA test, an examiner manually moves the subject's head from side to side. Not only may there be some level of resistance of the passive head rotation, but continuous movement at the necessary rate of rotation cannot be ensured, resulting in potentially inaccurate assessment results.

The disclosure is based, in part, on the inventors' recognition that a portable computing device can be used to detect characteristics of movement of a subject's head, and present a plurality of optotypes on the display of the portable computing device when the movement meets pre-established criteria. In some embodiments, a pre-established criteria refers to a threshold (e.g., a movement type, minimum velocity, speed, frequency, etc.) a characteristic of head movement (e.g., rotational movement) meets, thereby initiating presentation of a plurality of optotypes on the display of a portable computing device. Examples of pre-established criteria include but are not limited to rotational movement (e.g., rotational movement at a frequency greater than 2 Hz), flexion movement (e.g., forward and backward movement of the head), extension/hyperextension movement, and abduction/adduction movement. In some embodiments, the pre-established criteria is rotational movement.

Standard DVA testing may also involve the use of a metronome for timing the frequency of head rotation from side to side (e.g., +/−30 degrees) at a minimum frequency of 2 Hertz (Hz) in effort to achieve the necessary rate of rotation (see, for example, Rine et al., Neurology (2003) 11 Supp 3: S25-31). In some embodiments of methods described herein, a subject's head rotation ranges from about 1 degree of rotation to about 180 degrees of rotation. In some embodiments, a subject's head rotation ranges from about 5 degrees of rotation to about 90 degrees of rotation (e.g., 5°, 10°, 15°, 20°, 25°, 30°, 35°, 40°, 45°, 50°, 55°, 60°, 65°, 70°, 75°, 80°, 85°, or 90°). In some embodiments, a subject's head rotation is more than 180° (e.g., 270°, 360°, etc.). In some embodiments, the frequency of a subject's head rotation ranges from about 2 Hz to about 10 Hz (e.g., about 2 Hz, 3 Hz, 4 Hz, 5 Hz, 6 Hz, 7 Hz, 8 Hz, 9 Hz, 10 Hz).

During the DVA assessment, the subject is presented an optotype, more than one optotype presented sequentially, or group of optotypes. As used herein, an “optotype” refers to a symbol or group of symbols that is presented to a subject so that a determination can be made as to whether the subject saw the symbol or groups of symbols correctly. Examples of optotypes include letters of an alphabet (e.g., English alphabet, Cyrillic alphabet, Greek alphabet, Arabic alphabet, etc.), symbols (e.g., mathematical symbols, scientific symbols, glyphs, etc.), or any combination of the foregoing. Following presentation of an optotype, the subject may indicate by any method the identity of the optotype he or she saw. The subject's response may be compared to the actual optotype and the accuracy of the response assessed. An optotype may be presented in any form, such as a single optotype or a group or plurality of optotypes. In some embodiments, a group of optotypes may be presented in the form of a vision chart, such as a Snellen chart, a LogMAR chart, LEA vision test, or a Landolt C test, or any other form known in the art.

Without wishing to be bound by any particular theory, optotypes can be presented in a variety of sizes. Optotype size can be measured by any suitable parameter, such as length (e.g., inches, centimeters, millimeters, etc.), font size (e.g., 9 point, 10 point, 12 point, 14 point, 16 point, etc.), and/or pixel size (e.g., pixel height). In some embodiments of methods described herein, the optotypes of the test are presented in decreasing size, becoming more difficult for the subject to accurately see and identify. In some embodiments of a DVA test described herein, the smallest optotype that is correctly identified by the subject is recorded and compared to the smallest optotype that is corrected identified by the subject in a static visual acuity (SVA) test. The difference in results from the DVA test compared to the responses from the SVA test for the subject is an indication the subject's DVA.

Additional DVA assessments include a computerized test, which requires the use of at least one computer or laptop, two monitors and a specially designed headset that includes an accelerometer. The headset detects head acceleration and commands the computer to only present optotypes on a monitor when the subject's head is moving at a specified rate (e.g., 2 Hz) to ensure proper vestibular stimulation.

### Static Visual Acuity (SVA)

The most common measure of visual acuity is static visual acuity (SVA). Conventional methods to assess SVA involve presenting optotypes to a subject at a set distance from the subject. The smallest optotype the subject correctly identifies determines the level of static visual acuity. A subject's results (e.g., smallest optotype correctly identified) from a DVA assessment may be compared to the results (e.g., smallest optotype correctly identified) from the subject in a SVA assessment to determine whether the subject has a defect in dynamic visual acuity.

### Oculomotor Function

The oculomotor system is a complex sensorimotor network which controls eye movements by incorporating volitional eye movements as well as visual information into appropriate motor outputs to the extra-ocular muscles to direct a one's gaze. Oculomotor behaviors may be indicators of neurological function. Two measures of oculomotor function are saccades and visual smooth pursuit. Saccades are rapid eye movements that allow the eyes to move accurately from one visual target to another, whereas smooth pursuit eye movements enable the eye to automatically track objects of interest as the object moves.

### Mobile Computing Device for Assessing DVA and SVA

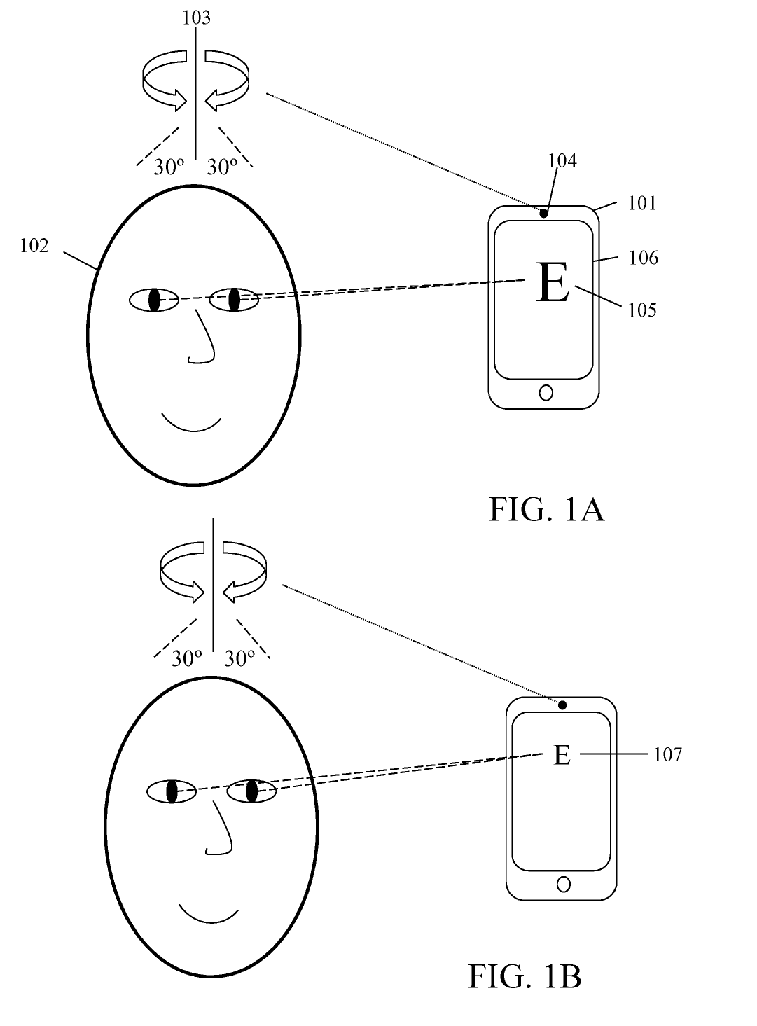

The methods and applications described herein provide a portable system for accurately assessing visual acuity including both dynamic and static acuity. FIG. 1 illustrates an example of a DVA assessment performed using a portable computing device such as a smart phone or related device. As demonstrated in FIG. 1A, the portable computing device (101) is placed in the visual field of the subject (102), and the subject rotates his or her head from side to side (along the yaw axis). The characteristics of head movement of the subject, such as the rotational movement (103), are detected by a sensor (104) on the portable computing device. When the characteristics of head movement meet pre-established criteria, at least one optotype (105) is presented on the display (106) of the portable computing device. In some embodiments, the pre-established criteria is the rotational movement of the subject's head at an appropriate rate (e.g., at least 2 Hz). In some embodiments, the pre-established criteria is the acceleration or position of the subject's head. The subject identifies the optotype he or she perceived and the subject's response is input into the application. This information may be input into the application.

Portable computing device may be programmed to receive input from the subject through any suitable interface. For example, the optotype may correspond to a character that appears on a keyboard, and the user may indicate the optotype perceived by striking a key on the keyboard. Alternatively, the computing device may receive the user input through a microphone, configured to implement a voice recognition input. In yet other embodiments, the input may be received through a user making a gesture on a touch screen. For example, the user may trace out a pattern representing the optotype or a feature of the optotype. As a specific example, in embodiments in which the optotype is represented by the orientation of a character, the user may indicate the optotype by swiping in a direction corresponding to the perceived orientation. In some embodiments, the user indicates the optotype by voice recognition of the optotype. In some embodiments, the user indicates the optotype by tilting the device in a specified direction.

As shown in FIG. 1B, additional optotypes of decreasing size (107) can be subsequently presented to the subject. The subject also identifies the subsequently presented optotypes he or she perceives, and the response(s) are input into the application. The subject's response in identification of each optotype is compared to the actual identity of the optotype and evaluated as to whether the subject's response is correct or incorrect. The smallest optotype that is accurately identified is recorded and may be compared to the smallest optotype that is accurately identified in a SVA assessment.

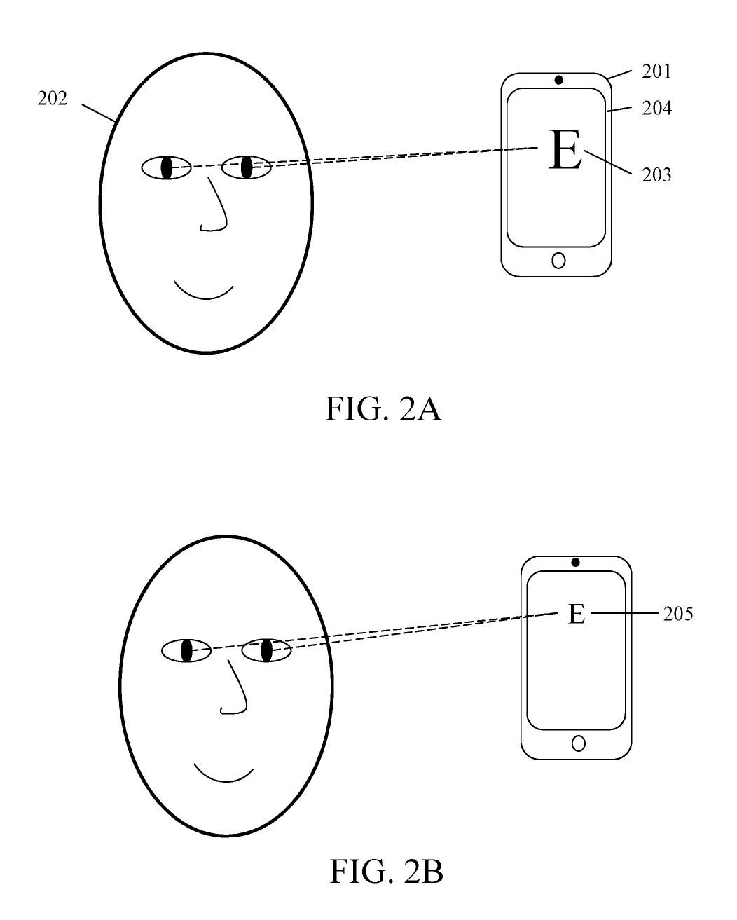

FIG. 2 illustrates an example of a SVA assessment performed using a portable computing device such as a smart phone or related device. As demonstrated in FIG. 2A, the portable computing device (201) is placed in the visual field of the subject (202). In contrast to the DVA assessment, the subject's head is maintained stationary for the SVA assessment. At least one optotype (203) is presented on the display of the portable computing device (204). The subject identifies the optotype he or she perceived and the subject's response is input into the application. As shown in FIG. 2B, additional optotypes of decreasing size (205) can be subsequently presented to the subject. The subject also identifies the subsequently presented optotypes he or she perceives, and the response(s) may be input into the application. The subject's response in identification of each optotype is compared to the actual identity of the optotype and evaluated as to whether the subject's response is correct or incorrect. The smallest optotype that is accurately identified is recorded and may be compared to the smallest optotype that is accurately identified in a DVA assessment.

In some embodiments, a difference between the smallest optotype correctly identified in the DVA assessment is compared to the smallest optotype identified in the SVA assessment. A difference above a threshold may indicate an abnormal result (e.g., the subject has a disorder such as a brain tumor/lesion, concussion, or other neurological disease). For example, in some embodiments a difference of 3 or less optotype sizes (e.g., no difference, 1, 2, or 3 size differences) may indicate a “normal” result, and the subject has normal (no disorder) DVA. In some embodiments, a difference of more than 3 optotype sizes (e.g., 4, 5, 6, 7, or more size differences) indicates an “abnormal” result. In some embodiments, an abnormal result indicates the subject likely has a VOR defect or disorder. In some embodiments the optotype is presented from a visual acuity chart, such as an ETDRS chart or a LeaSymbols chart.

### Computing Device for Assessing Saccades and Smooth Pursuit

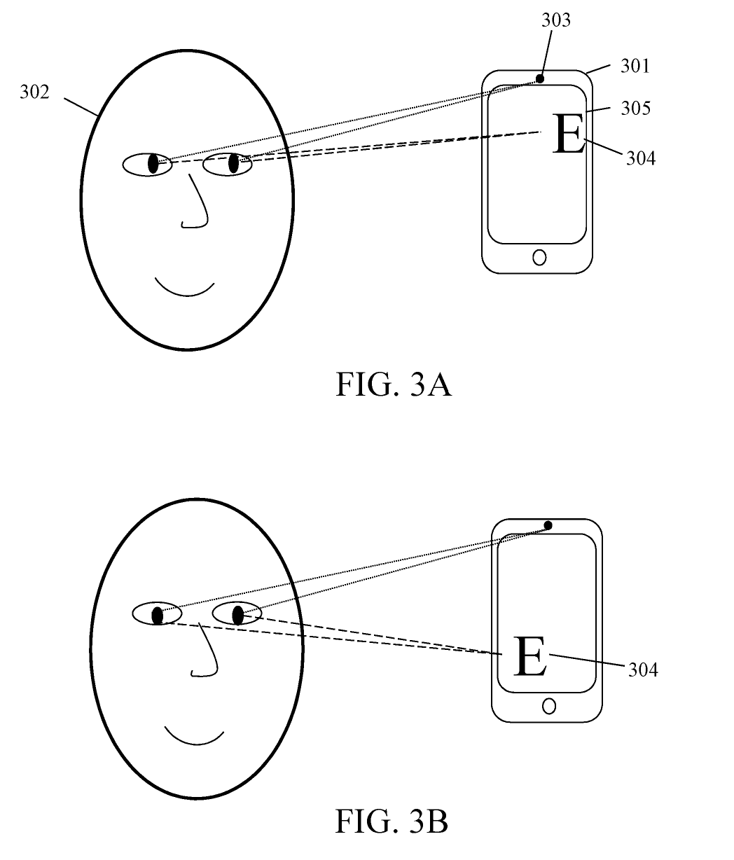

The methods and applications described herein provide a portable system for accurately assessing oculomotor function, including saccades and smooth pursuit. FIG. 3 illustrates an example of a saccades assessment performed using a portable computing device such as a smart phone or related device. As demonstrated in FIG. 3A, the portable computing device (301) is placed in the visual field of the subject (302). An optotype (304) is presented on the display (305) of the portable computing device. In some embodiments, the subject may identify the optotype he or she perceives and the subject's response may be input into the application. The subject's eye movement and rate or speed of movement is detected by a sensor (303) on the portable computing device, defining a first trajectory. As shown in FIG. 3B, additional optotypes (e.g., (304)) in different locations on the display of the portable computing device are subsequently presented to the subject and the first optotype is removed. The subject's eye movement and rate or speed of movement to the subsequent optotype is detected by a sensor (303) on the portable computing device, defining a second trajectory. The correlation between the first trajectory and the second trajectory is determined. Eye movement that occurs in less than 100 milliseconds (ms) is defined as a saccade.

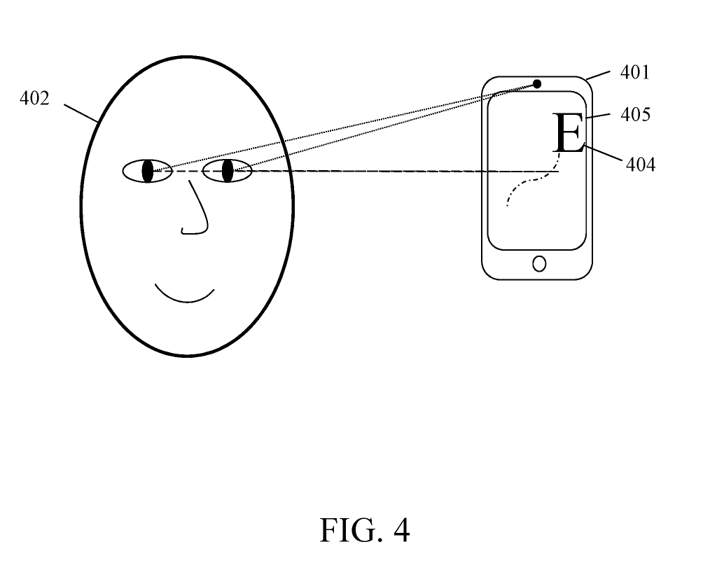

FIG. 4 illustrates an example of a smooth pursuit assessment performed using a portable computing device such as a smart phone or related device. As demonstrated in FIG. 4, the portable computing device (401) is placed in the visual field of the subject (402). An optotype (404) is presented in a location on the display (405) of the portable computing device. In contrast to the saccades assessment in which the optotypes are static on the display and the one or more additional optotypes are presented while the first optotype is removed, in the smooth pursuit assessment, the first optotype is not static but rather moves on the display of the portable computing device. The subject's eye movement and rate of movement following the moving optotype is detected by a sensor (403) on the portable computing device. Eye movement that occurs in more than 100 milliseconds (ms) is defined as a smooth pursuit.

In performing any of the methods described herein, the computing device may be positioned in the subject's field of vision. In some embodiments, the computing device is maintained at a position in the subject's field of vision at a distance between 2 inches and 20 feet from the subject. In some embodiments, the distance is between 6 inches and 15 feet, 1 foot and 10 feet, 2 feet and 8 feet, or between 4 feet and 6 feet from the subject. In some embodiments, the distance is between 1-2 feet, 2-3 feet, 3-4 feet, 4-5 feet, 5-6 feet, 6-7 feet, 7-8 feet, 8-9 feet, 9-10 feet, 10-11 feet, 11-12 feet, 12-13 feet, 13-14 feet, 14-15 feet, 15-16 feet, 16-17 feet, 17-18 feet, 18-19 feet, or between 19-20 feet from the subject. In some embodiments, the distance is 2, 3, 4, 5, 6, 7, 8, 9, 10, 11, 12 inches from the subject. In some embodiments, the distance is 1, 2, 3, 4, 5, 6, 7, 8, 9, 10, 11, 12, 13, 14, 15, 16, 17, 18, 19, or 20 feet from the subject.

The methods described herein may provide results for analysis by a clinician, such as a physician. In some embodiments, the results include the average latency (in ms), average peak velocity (degrees/second), number of catch-up saccades, phase lead (degrees), phase lag (degrees), number of over-shoots (hypermetric saccades, number of under-shoots (hypometric saccades), and saccade gain (ratio of average eye movement/target movement). Any of the results obtained using the methods and storage media described herein may be used to assess a condition in the subject and/or progression of the condition. In some embodiments, the results are used to diagnose the subject.

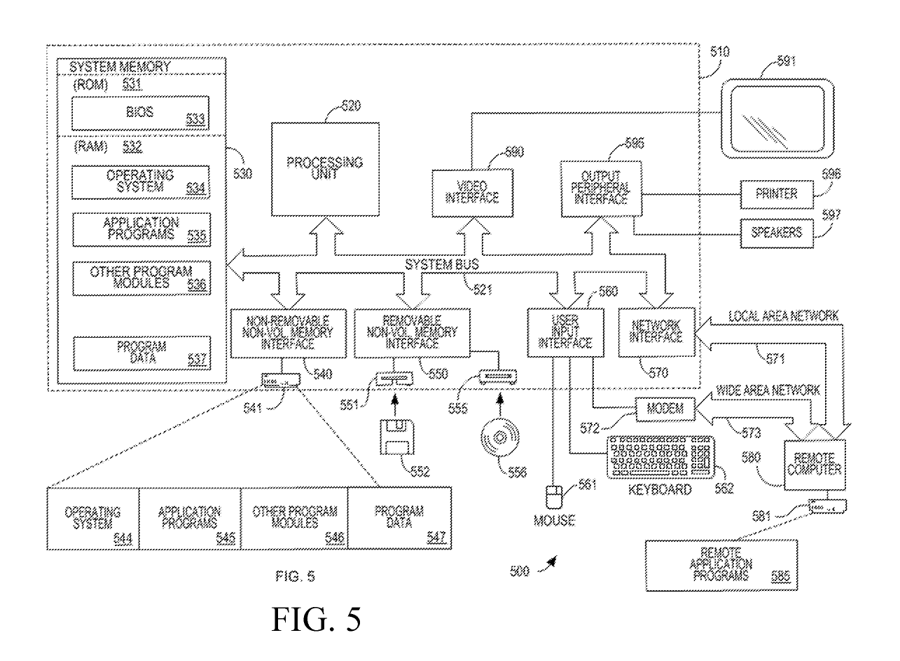

FIG. 5 illustrates an example of a suitable computing system environment 500 on which the invention may be implemented. The computing system environment 500 is only one example of a suitable computing environment and is not intended to suggest any limitation as to the scope of use or functionality of the invention. Neither should the computing environment 500 be interpreted as having any dependency or requirement relating to any one or combination of components illustrated in the exemplary operating environment 500.

The invention is operational with numerous other general purpose or special purpose computing system environments or configurations. Examples of well-known computing systems, environments, and/or configurations that may be suitable for use with the invention include, but are not limited to, personal computers, server computers, hand-held or laptop devices, smartphones, tablets, multiprocessor systems, microprocessor-based systems, set top boxes, programmable consumer electronics, network PCs, minicomputers, mainframe computers, distributed computing environments that include any of the above systems or devices, and the like. Some of the elements illustrated in FIG. 5 may not be present, depending on the specific type of computing device. Alternatively, additional elements may be present in some implementations.

The computing environment may execute computer-executable instructions, such as program modules. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 5, an exemplary system for implementing the invention includes a general purpose computing device in the form of a computer 510. Components of computer 510 may include, but are not limited to, a processing unit 520, a system memory 530, and a system bus 521 that couples various system components including the system memory to the processing unit 520. The system bus 521 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 510 typically includes a variety of computer readable media. Computer readable media can be any available media that can be accessed by computer 510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 510. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 530 includes computer storage media in the form of volatile and/or nonvolatile memory such as read only memory (ROM) 531 and random access memory (RAM) 532. A basic input/output system 533 (BIOS), containing the basic routines that help to transfer information between elements within computer 510, such as during start-up, is typically stored in ROM 531. RAM 532 typically contains data and/or program modules that are immediately accessible to and/or presently being operated on by processing unit 520. By way of example, and not limitation, FIG. 5 illustrates operating system 534, application programs 535, other program modules 536, and program data 537.

The computer 510 may also include other removable/non-removable, volatile/nonvolatile computer storage media. By way of example only, FIG. 5 illustrates a hard disk drive 541 that reads from or writes to non-removable, nonvolatile magnetic media, a magnetic disk drive 551 that reads from or writes to a removable, nonvolatile magnetic disk 552, and an optical disk drive 555 that reads from or writes to a removable, nonvolatile optical disk 556 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 541 is typically connected to the system bus 521 through an non-removable memory interface such as interface 540, and magnetic disk drive 551 and optical disk drive 555 are typically connected to the system bus 521 by a removable memory interface, such as interface 550.

The drives and their associated computer storage media discussed above and illustrated in FIG. 5, provide storage of computer readable instructions, data structures, program modules and other data for the computer 510. In FIG. 5, for example, hard disk drive 541 is illustrated as storing operating system 544, application programs 545, other program modules 546, and program data 547. Note that these components can either be the same as or different from operating system 534, application programs 535, other program modules 536, and program data 537. Operating system 544, application programs 545, other program modules 546, and program data 547 are given different numbers here to illustrate that, at a minimum, they are different copies. A user may enter commands and information into the computer 510 through input devices such as a keyboard 562 and pointing device 561, commonly referred to as a mouse, trackball or touch pad. Other input devices (not shown) may include a microphone, joystick, game pad, satellite dish, scanner, or the like. These and other input devices are often connected to the processing unit 520 through a user input interface 560 that is coupled to the system bus, but may be connected by other interface and bus structures, such as a parallel port, game port or a universal serial bus (USB). A monitor 591 or other type of display device is also connected to the system bus 521 via an interface, such as a video interface 590. In addition to the monitor, computers may also include other peripheral output devices such as speakers 597 and printer 596, which may be connected through a output peripheral interface 595.

The computer 510 may operate in a networked environment using logical connections to one or more remote computers, such as a remote computer 580. The remote computer 580 may be a personal computer, a server, a router, a network PC, a peer device or other common network node, and typically includes many or all of the elements described above relative to the computer 510, although only a memory storage device 581 has been illustrated in FIG. 5. The logical connections depicted in FIG. 5 include a local area network (LAN) 571 and a wide area network (WAN) 573, but may also include other networks. Such networking environments are commonplace in offices, enterprise-wide computer networks, intranets and the Internet.

When used in a LAN networking environment, the computer 510 is connected to the LAN 571 through a network interface or adapter 570. When used in a WAN networking environment, the computer 510 typically includes a modem 572 or other means for establishing communications over the WAN 573, such as the Internet. The modem 572, which may be internal or external, may be connected to the system bus 521 via the user input interface 560, or other appropriate mechanism. In a networked environment, program modules depicted relative to the computer 510, or portions thereof, may be stored in the remote memory storage device. By way of example, and not limitation, FIG. 5 illustrates remote application programs 585 as residing on memory device 581. It will be appreciated that the network connections shown are exemplary and other means of establishing a communications link between the computers may be used.

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the invention will include every described advantage. Some embodiments may not implement any features described as advantageous herein and in some instances. Accordingly, the foregoing description and drawings are by way of example only.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present invention as discussed above. As used herein, the term “computer-readable storage medium” encompasses only a computer-readable medium that can be considered to be a manufacture (i.e., article of manufacture) or a machine. Alternatively or additionally, the invention may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. At least one non-transitory computer storage medium comprising computer executable instructions that, when executed on a portable computing device, controls the computing device to perform a method of assessing dynamic visual acuity (DVA) of a subject, the method comprising:
deriving from sensor data characteristics of head movement of the subject;
for each of a plurality of optotypes, the plurality of optotypes having different sizes:
when the characteristics meet pre-established criteria, presenting an optotype of the plurality of optotypes on a display of the portable computing device;
receiving input indicating the subject's perception of the displayed optotype;
based on a comparison of displayed optotypes and received input, recording a smallest optotype of the plurality of optotypes that was accurately reported by the subject

2. The non-transitory computer storage medium of claim 1, wherein the smallest optotype of the plurality of optotypes accurately reported by the subject is compared to a smallest optotype of the plurality of optotypes accurately reported by the subject in a static visual acuity assessment.

3. The non-transitory computer storage medium of claim 1 or 2, wherein the difference between the smallest optotype accurately reported by the subject in the dynamic visual acuity assessment and the smallest optotype accurately reported by the subject in the static visual acuity assessment of 2 or fewer optotype sizes indicates a normal result.

4. The non-transitory computer storage medium of claim 2, wherein the method further comprises indicating an abnormal result in response to the difference between the smallest optotype accurately reported by the subject in the dynamic visual acuity assessment and the smallest optotype accurately reported by the subject in the static visual acuity assessment being more than 3 optotype sizes.

5. The non-transitory computer storage medium of any one of claims 1 to 4, wherein the pre-established criteria of the characteristics of head movement of the subject is rotational movement.

6. The non-transitory computer storage medium of claim 5, wherein the rotational movement is at a frequency of about 2 Hertz.

7. The non-transitory computer storage medium of any one of claims 1 to 6, wherein the sensor data comprises images acquired by a camera on the portable computing device.

8. The non-transitory computer storage medium of any one of claims 1 to 7, wherein the input is received using a method selected from the list consisting of by video analysis, accelerometer input, manual touch insertion using a touch screen, keyboard, drop down menu, and toggle menu.

9. A method for performing a dynamic visual acuity assessment, comprising
positioning a portable computing device in the visual field of a subject at a distance from the subject;
detecting movement subject's head;
when the subject's head movement meets pre-established criteria, presenting a plurality of optotypes on the display of the portable computing device;
receiving information indicating the subject's perception of the displayed optotypes;
based on a comparison of displayed optotypes and received input, recording a smallest optotype of the plurality of optotype that was accurately reported by the subject.

10. The method of claim 9, wherein the distance is between 2 inches and 20 feet from the subject.

11. The method of claim 9 or 10, wherein the smallest optotype of the plurality of optotypes accurately reported by the subject is compared to a smallest optotype of the plurality of optotypes accurately reported by the subject in a static visual acuity assessment.

12. The method of claim 11, wherein the difference between the smallest optotype accurately reported by the subject in the dynamic visual acuity assessment and the smallest optotype accurately reported by the subject in the static visual acuity assessment of 3 or fewer optotype sizes indicates a normal result.

13. The method of claim 11, wherein the method further comprises indicating an abnormal result in response to the difference between the smallest optotype accurately reported by the subject in the dynamic visual acuity assessment and the smallest optotype accurately reported by the subject in the static visual acuity assessment being more than 2 optotype sizes.

14. The method of any one of claims 9 to 13, wherein the portable computing device is maintained in a static position in the subject's field of vision.

15. The method of any one of claims 9 to 14, wherein the pre-established criteria of the characteristics of head movement of the subject is rotational movement.

16. The method of claim 15, wherein the rotational movement is at a frequency of about 2 Hertz.

17. The method of any one of claims 9 to 16, wherein the sensor data comprises images acquired by a camera on the portable computing device.

18. The method of any one of claims 9 to 17, wherein the input is received using a method selected from the list consisting of by video analysis, accelerometer input, manual touch insertion using a touch screen, keyboard, drop down menu, and toggle menu.

19. At least one non-transitory computer storage medium comprising computer executable instructions that, when executed on a portable computing device, controls the computing device to perform a method of assessing saccades of a subject, the method comprising:
presenting on a display of the portable computing device at least one optotype in different locations at different times thereby defining on the display a first trajectory;
processing sensor data to determine movement of the eyes and speed of movement, thereby defining a second trajectory;
determining correlation between the first trajectory and the second trajectory.

20. The non-transitory computer storage medium of claim 19, wherein a duration of eye movement from the optotype at a first location to the optotype at a second location of less than 100 ms indicates a saccade.

21. A method for performing a saccade assessment comprising
positioning a portable computing device in the visual field of a subject at a distance from the subject;
presenting on a display of the portable computing device at least one optotype in different locations at different times thereby defining on the display a first trajectory;
detecting movement of the subject's eyes and the speed of movement;
receiving information indicating movement of the subject's eyes and speed of the movement, thereby determining a second trajectory;
determining the correlation between the first trajectory and the second trajectory.

22. The method of claim 21, wherein the portable computing device is maintained in a static position in the subject's field of vision.

23. The method of claim 21 or 22, wherein the movement of the subject's eyes and the speed of movement is detected by a camera on the portable computing device.

24. The method of any one of claims 21 to 23, wherein the position in the subject's field of vision is at a distance between 2 inches and 20 feet from the subject.

25. At least one non-transitory computer storage medium comprising computer executable instructions that, when executed on a portable computing device, controls the computing device to perform a method of assessing smooth pursuits of a subject, the method comprising:
presenting on a display of the portable computing device a moving optotype;
processing sensor data to determine movement of the subject's eyes and speed of movement.

26. The method of claim 25, wherein the portable computing device is maintained in a static position in the subject's field of vision.

27. The method of claim 25 or 26, wherein the movement of the subject's eyes and the speed of movement is detected by a camera on the portable computing device.

28. The method of any one of claims 25 to 27, wherein the position in the subject's field of vision is at a distance between 2 inches and 20 feet from the subject.
